(19) United States
(12) Patent Application Publication
(10) Pub. No.: US 2021/0137602 A1
(43) Pub. Date: May 13, 2021

(54) METHOD TO PRECISELY PLACE VERTEBRAL PEDICLE ANCHORS DURING SPINAL FUSION SURGERY

(71) Applicant: Cutting Edge Surgical, Inc., Gilbertsville, PA (US)

(72) Inventor: Mark Goodwin, Gilbertsville, PA (US)

(21) Appl. No.: 17/092,401

(22) Filed: Nov. 9, 2020

Related U.S. Application Data
(60) Provisional application No. 62/932,689, filed on Nov. 8, 2019.

(51) Int. Cl. A61B 34/10; A61B 5/00; A61F 2/44; A61F 2/46; G16H 20/40; G16H 30/20

(52) U.S. Cl. CPC A61B 34/10; A61B 2090/3762; A61B 5/4566; A61F 2/4455; A61F 2/4611; A61B 5/7415; A61B 5/742; A61B 5/4504; A61B 5/004; G16H 20/40; G16H 30/20; A61B 2505/05; A61B 2034/107; A61F 2002/4633; A61F 2002/4662; A61B 5/0095

(57) ABSTRACT

Disclosed are methods to locate the posterior cancellous os of a pedicle on a vertebra and/or optimize implant trajectory in a subject, especially for spinal fusion surgery. The methods generally include: (a) directing a beam of laser light at or positioning a fiber that diffuses laser light over a pars interarticularis of the vertebra of the subject; and (b) acquiring photoacoustic signal for imaging the vertebra. The methods can further include selecting and/or adjusting parameters of the laser light such that the laser light penetrates a single layer of cortical bone covering the pars interarticularis into cancellous bone thereunder, reaching a quantifiable depth within the cancellous bone. Preferably, the quantifiable depth reaches at least about the full length of the pedicle.

### CROSS REFERENCE TO RELATED APPLICATIONS

This application claims benefit of U.S. Provisional Application No. 62/932,689, filed Nov. 8, 2019, hereby incorporated herein by reference in its entirety.

### FIELD OF THE INVENTION

The invention generally relates to the field of medicine and more specifically to methods to optimize placement of spinal implants during spinal fusion surgery.

### BACKGROUND OF THE INVENTION

Many skeletal conditions require the placement of implants, which must be correctly positioned to optimally address the surgical condition. An example of the challenges encountered during skeletal surgery is demonstrated in reconstructive surgery of the spine, where metallic, synthetic, or bioactive implants, hereafter “implants”, are placed to assist in the healing of a bony fusion or to correct and/or stabilize the deformed or disrupted vertebral column. During complex spinal surgery, e.g., fusion, implants are often placed in the vertebral pedicle of the spine vertebra, a narrow column of bone that connects the dorsal (back) part of the spinal to the vertebral body (front). Although the pedicle is an excellent anchor point from a biomechanical perspective, delicate tissues including the spinal cord, nerve roots and major blood vessels (aorta and vena cava) surround it. All of these structures can be injured during placement, leading to catastrophic consequences of the patient and with associated medico-legal implications.

Current surgical technique for the placement of implants into the vertebral pedicle involves the placement of a pilot hole over the top of the column of the vertebral pedicle, followed by cannulation (creation of a bony passage) with a blunt instrument prior to placing the pedicle implant. To perform this procedure correctly, a surgeon must determine the point to open on the surface of the bone composed of a dense cortical-bone layer directly over the vertebral pedicle os composed of cancellous bone, and then pass the instruments used to open a hole or passage through the vertebral pedicle along a correct trajectory from back to front through the softer cancellous bone core of the pedicle column. If the starting point is not accurate, there is a high risk that a false passage will be created thereby disrupting or breaching the column of cortical bone needed to maintain intra-cancellous passage of the implant. If the implant passage is extra-cortical, it may damage critical structures like spinal cord, nerves, and/or blood vessels. Even if these structures are not directly insulted by an extra-cortical implant, its mere proximity to them may elicit chronic pain.

To assist spinal surgeons in finding the pedicle, conventional radiographic imaging technologies have been used, the most basic of which is a fluoroscopic “C-Arm”. This portable x-ray unit can be used to visualize the two dimensional anatomy of the spine in relation to the instrument that the surgeon is using to make the pedicle passage. However, the use of radiographic imaging provides only a two-dimensional picture of the complex three-dimensional anatomy of the spine and exposes the surgical team and patient to potentially large amounts of ionizing radiation. In addition, the equipment is bulky and cumbersome to use, and requires a dedicated technician to operate.

More sophisticated imaging techniques have been developed to assist with spinal implant placement, including computer-assisted image-guided surgery platforms, manually or robotically controlled. These systems use pre-acquired images, not real-time images, from fluoroscopy, ultrasound, or computed axial tomography (CAT) scanning that, in combination with software, can be correlated to the patient's anatomy during surgery. To accomplish this, a reference array must be securely attached to the patient's anatomy and to any instruments used for the pilot hole and cannulation phase of the surgery.

Although this high-tech approach seems appealing, the use of computer-assisted guidance platforms during spinal surgery has been fraught with difficulties that have limited their use. For example, the setup and use of the equipment is cumbersome and highly technical. Additionally, the equipment is bulky and sensitive to being accidentally “bumped” during the procedure, dislodging the reference array attached to the spine thereby rendering the navigation unreliable and inaccurate. More recently, robotic platforms have been introduced. Unfortunately, all robotic platforms are driven by the same “virtual guidance” used in “manual” image-guided navigation. While there is arguably incremental benefit, it is outweighed by a very high cost in dollars, time added to the procedure, and ionizing radiation. Moreover, as new surgical techniques evolve, e.g., “XLIF”, the application of robots to patient anatomy may be difficult or impossible. Surgeons have found the lack of real-time data prevents them from routinely trusting the navigated images for the placement of complex implant constructs.

An alternative to ionizing-radiation guidance platforms are hand-held real-time devices that rely solely on ultrasound or electrical bone impedance. For example, U.S. Pat. Nos. 6,579,244 and 6,849,047 describe devices for guidance, which create a channel for deployment of a pedicle implant. U.S. Pat. No. 6,719,692 describes a hollow drill or cutting instrument integrated with ultrasound that allows imaging during cutting. The only commercially available non-ionizing-radiation-based, hand-held guidance device is PEDIGUARD® which relies upon changes in bone impedance to limit the risk of a “full thickness” extra-cortical bone breach while being used to create a pedicle channel See Bolger et al., Eur Spine J, 2007, 16(11):1919-24. However, the utility of all these devices generally depends on access to the pedicle after the cortical bone covering the pedicle os has been disrupted and removed.

While experienced spine surgeons have a good understanding of complex anatomy, studies have documented a significant percentage of spinal screws lacking proper placement. Reasons for incorrect placement of pedicle implants include variations in spinal anatomy between individuals, altered spinal anatomy as a result of disease, trauma, or deformity of the spine, poor or misleading radiographic images of the spine, small pedicles, obesity, bony overgrowths from the joint obscuring the starting point, and/or poor bone quality. These factors can make the identification of the pedicle starting point and trajectory difficult to perform even by experienced spinal surgeons.

Accurate identification of the pedicle starting point is a key to successful and time-efficient navigation and arguably the “Achilles' heel” of spinal fusion surgery today. Therefore, there remains a need for methods to locate the pedicle starting point and/or optimize implant trajectory prior to bone breach in real time and without ionizing radiation.

It is an object of the present invention to provide a method to precisely locate a vertebral pedicle starting point, before disrupting the cortical bone layer, for accurate and safe placement of vertebral pedicle anchors in real time, without ionizing radiation. It is another object of the present invention to provide a method to define a path or trajectory through the pedicle os and insure creation of an intra-cancellous channel from the pedicle os to deep within the cancellous bone of the anterior vertebral body.

### BRIEF SUMMARY OF THE INVENTION

Disclosed are methods to non-invasively locate an intra-cancellous pedicle implant path with a non-ionizing form of imaging and/or tissue delineation for placement of an implant, such as a pedicle anchor, in real time for spinal fusion surgery.

The methods generally include: (a) directing a beam of laser light at or positioning a fiber that diffuses homogenous laser light preferably over the region of pars interarticularis of a vertebra of a subject; and (b) acquiring photoacoustic signal for imaging the vertebra. Anatomically, the pars interarticularis is a reliable posterior vertebral landmark, from which to initiate laser interrogation of cortical bone especially since other prominent posterior structures like the superior articular facet may be obscured or obliterated by the effects of spondylosis, trauma, deformity, etc. Preferably, the methods do not include directing a beam of laser light at or positioning a fiber that diffuses homogenous laser light over the superior articular facet, in order to locate the placement site of the implant.

In some embodiments, the methods further include and/or adjusting parameters of the laser light such that the laser light penetrates a single layer of cortical bone overlying the pedicle os and into the cancellous bone thereunder, reaching a quantifiable depth within the cancellous bone. In some embodiments, the quantifiable depth covers at least the full length of the pedicle. In some embodiments, the quantifiable depth extends beyond the length of the pedicle and further into the cancellous bone of the anterior vertebral body.

In some embodiments, the methods further include locating an intra-cancellous pedicle implant path based on the photoacoustic signal from step (b). The intra-cancellous pedicle implant path starts from an intact cortical bone surface overlying the pedicle os, and extends through the cancellous bone of the pedicle into the cancellous bone of the anterior vertebral body of the vertebra. In some embodiments, the intra-cancellous pedicle implant path is pre-determined by a computed tomography scan, such as a computed axial tomography scan, prior to step (a).

In some embodiments, the methods further include selecting and/or adjusting parameters of the laser light to penetrate surrounding soft tissues of the vertebra (especially around the pars interarticularis region), including skin, adipose, connective tissues, muscle, tendons, ligaments, etc., for tissue delineation. This can provide valuable information to identify the precise location of the pedicle os.

In some embodiments, the laser wavelength is selected as the absorption wavelength of an inorganic constituent of bone, such as hydroxyapatite and calcium phosphate.

In some embodiments, the laser parameters such as wavelength, fluence, and frequency can be selected to interact preferentially with endogenous elements of one or more of the surrounding soft tissues, e.g., oxyhemoglobin, deoxyhemoglobin, lipids, water, etc., or exogenous elements, the so-called “contrast agents”, e.g., methylene blue, indocyanine green (ICG), nanoparticles, etc., to alter the quality of the photoacoustic signal in predictable and quantifiable ways, such as depth of penetration, spatial resolution, etc.

In some embodiments, the methods further include, after step (b), transmitting the photoacoustic signal to a visual form on a monitor, and/or an audible form, which optionally changes pitch and/or volume based upon proximity of the implant to the pedicle os for guiding surgical operation.

Unless defined otherwise, all technical and scientific terms used herein have the same meanings as commonly understood by one of skill in the art to which the disclosed invention belongs. Those skilled in the art will recognize, or be able to ascertain using no more than routine experimentation, many equivalents to the specific embodiments of the invention described herein. Such equivalents are intended to be encompassed by the claims.

Additional advantages of the disclosed methods will be set forth in part in the description which follows, and in part will be understood from the description, or can be learned by practice of the disclosed methods. The advantages of the disclosed methods will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods, and together with the description, serve to explain the principles of the disclosed methods.

FIG. 1 shows an anatomy diagram (axial view) of an exemplary human vertebra, wherein different vertebral structures are illustrated. The pedicles are circled by solid lines, and the laminas are circled by dotted lines.

FIGS. 2A and 2B show anatomy diagrams (dorsal view and lateral view, respectively) of an exemplary human vertebra, wherein the region of pars interarticularis is circled by dashed line and the pedicle is circled by solid line. The longest anterior-posterior “column of cancellous bone” (the cylinder outlined by dotted line) in any human vertebrae is that which embodies cancellous bone from all three vertebral columns of bone, i.e., anterior, middle, and posterior. All human vertebrae, with perhaps the exception of Cl (a.k.a., atlas), include two vertebral pedicles.

FIGS. 3A and 3B show anatomy diagrams (axial view and lateral view, respectively) of an exemplary human vertebra implanted with a pair of pedicle anchors, i.e., titanium screws, across vertebral structures comprising the posterior, middle, and anterior spinal columns. Optimal intra-cancellous cannulation paths incorporate the foregoing spinal columns FIG. 4 shows an anatomy diagram of a lumbar vertebra with the cortical bone layer removed. The longest uninterrupted “column of cancellous bone” derives from posterior, middle, and anterior column structures as denoted by the cylinder diagram therein.

FIG. 5 shows a computed axial tomography image of a lumbar vertebra. The double-headed arrows denote PAi image parameters: the vertical arrow denotes minimum depth of PAi penetration for locating the intra-cancellous pedicle implant path; the horizontal arrow denotes intra-cancellous pedicle width. The area captured by the cylinder (dashed lines) represents the column of cancellous bone in the pedicle. The area circled by the solid line demarcates the region of the cancellous bone in the vertebra. The “white” signals outside the outlined region of cancellous bone correspond to the cortical bone covering the vertebra.

FIG. 6 illustrates laser light positioning for PAi imaging of an exemplary human vertebra. In the anatomy diagram (axial view) of the vertebra, the region of pars interarticularis is circled by dashed line. With the pars interarticularis as a landmark, diffused laser light (represented by the oval-shaped dotted circle) or laser beam (represented by the black dot) is manipulated until a signal or visual display confirms a top layer of cortical bone followed by an uninterrupted column of cancellous bone to a depth at or beyond the full range of the pedicle.

FIG. 7 illustrates laser orientation (trajectory) for PAi imaging of an exemplary human vertebra. In the anatomy diagram (lateral view) of the vertebra, the region of pars interarticularis is circled by dotted line, and the region of pedicle is circled by solid line. Exemplary laser beams with different trajectories are illustrated by arrows; all of the laser beams are directed at the region of pars interarticularis. The solid arrows illustrate the trajectories of the laser beams before they arrive at the cortical bone covering the region of pars interarticularis. The dash-dotted arrows illustrate the continued trajectories of the laser beams within the cancellous bone of the vertebra.

FIGS. 8A and 8B show an exemplary vertebra and its corresponding computed axial tomography image, respectively. The double-headed arrow between “A” and “B” illustrate the minimum depth of PAi penetration for locating the intra-cancellous pedicle implant path. The double-headed arrow between “C” and “D” illustrates the intra-cancellous pedicle width.

### DETAILED DESCRIPTION OF THE INVENTION

The disclosed methods can be understood more readily by reference to the following detailed description of particular embodiments and the examples included therein and to the drawings and their previous and following description. All methods described herein can be performed in any suitable order unless otherwise indicated or otherwise clearly contradicted by context.

The use of any and all examples or exemplary language (e.g., “such as”) provided herein, is intended merely to better illustrate the invention and does not pose a limitation on the scope of the invention unless otherwise claimed. No language in the specification should be construed as indicating any non-claimed element as essential to the practice of the invention.

Any discussion of documents, acts, devices, articles or the like which has been included in the present specification is not to be taken as an admission that any or all of these matters form part of the prior art base or were common general knowledge in the field relevant to the present disclosure as it existed before the priority date of each claim of this application.

### I. Definitions

The singular forms “a,” “an,” and “the” include plural reference unless the context clearly dictates otherwise.

The terms “may,” “may be,” “can,” and “can be,” and related terms are intended to convey that the subject matter involved is optional (that is, the subject matter is present in some embodiments and is not present in other embodiments), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

As used herein, “subject” includes, but is not limited to, human or non-human mammals. The term does not denote a particular age or sex. Thus, adult and newborn subjects, whether male or female, are intended to be covered. A patient refers to a subject afflicted with a disease or disorder. The term “patient” includes human and non-human mammal subjects.

As used herein, the term “pedicle” refers to either of two short cylindrical bony processes lying on either side of a vertebra that project posteriorly from the vertebral body and unite with the laminae to form a neural arch. Each pedicle has a superior and inferior notch that forms an intervertebral foramen with a pedicle on an adjacent vertebra, allowing for passage of spinal nerves and vessels. See FIG. 1 for an anatomical illustration of a pair of pedicles in a human vertebra. The term “pedicle os” refers to posterior cancellous origin of a pedicle.

As used herein, the term “pars interarticularis” refers to part of vertebra between inferior and superior articular process of the facet joint. Each vertebra has two regions of pars interarticularis (bilaterally). See FIGS. 2A and 2B for anatomical illustrations of pars interarticularis in a human vertebra.

### II. Methods

Disclosed hereunder is a detailed description of the methods to locate vertebral pedicle starting point, delineate surrounding soft tissues, optimize implant trajectory, and/or define the diameter of the cancellous core of the vertebral pedicle, especially for guiding spinal fusion surgery. The disclosed methods utilize morphologic features of all vertebral subtypes, i.e., lumbar, thoracic and cervical.

In preferred embodiments, the methods uses photoacoustic imaging (PAi) to locate one or more intra-cancellous pedicle implant path(s) in real time for placement of pedicle anchors for spinal fusion surgery in a subject. The methods include: (a) directing a beam of laser light at or positioning a fiber that homogenously diffuses laser light over the region of pars interarticularis of a vertebra of the subject; and (b) acquiring photoacoustic signal for imaging the vertebra.

In some embodiments, the methods further include and/or adjusting parameters of the laser light such that the laser light penetrates a single layer of cortical bone covering the pars interarticularis into cancellous bone thereunder, reaching a quantifiable depth within the cancellous bone. In some embodiments, the quantifiable depth covers at least the full length of the pedicle. In some embodiments, the quantifiable depth extends beyond the length of the pedicle and further into the cancellous bone of the anterior vertebral body.

In some embodiments, the methods further include locating an intra-cancellous pedicle implant path based on the photoacoustic signal from step (b). The intra-cancellous pedicle implant path starts from an intact cortical bone surface overlying the pedicle os, and extends through the cancellous bone of the pedicle into the cancellous bone of the anterior vertebral body. In some embodiments, the intra-cancellous pedicle implant path is constructed based upon a pre-measured line segment from a pre-op CT scan.

1. Spinal Fusions

Spinal fusion is a commonly practiced neurosurgical or orthopedic technique to join two or more unstable vertebrae at any level of the spine, cervical, thoracic, or lumbar. The relative instability of one vertebra relative to another may be caused by a variety of factors, i.e., disease, trauma, deformity, or surgery. Left untreated spinal instability may cause pain, neurologic compromise, or both. Surgeons set up a fusion by surgically introducing autograft or allograft between and/or around the affected vertebrae with the goal of graft incorporation by and between them. The resulting bony union prevents asynchronous motion between them and hopefully the pain or neurologic compromise related thereto. Since the process of graft incorporation can take many months it is essential the affected segment(s) are completely immobilized until they heal, i.e., a solid union is formed. This typically requires external or internal fixation of the affected segments by the use of rigid implants to form an immobile construct. The strength and stability of these constructs rely heavily on material strength of the implant and the strength of bone into which they are introduced. A single motion segment, i.e., two vertebrae, typically requires a minimum of two screws per vertebra and two rods to connect each lateral pair of screws. The strongest anatomic vertebral structure in which to anchor screw implants are the vertebral pedicles. With the exception of perhaps Cl (aka, Atlas), all human vertebra are comprised of two pedicles.

Examples of pedicle implants and systems thereof are known. See, for example, U.S. Pat. Nos. 6,488,681, 6,423,065, 6,312,431, 6,858,030, 7,163,539, 7,311,713, and patents cited therein. Conventional pedicle cannulation is essentially a “blind procedure”, meaning that the surgeon cannot visualize the starting point or passage of the instrument during the process. Instead, the surgeon must rely on a combination of an understanding of the normal spinal anatomy plus tactile feedback to achieve correct placement of the implant.

There is a strong correlation between mechanical construct strength and successful bony unions. Therefore, the least disruption to the vertebral pedicle while placing the implant the better. The histology of vertebral pedicles is comprised of a central core of cancellous bone which is spongy, blood-rich, and of variable diameter and density, contingent upon vertebral level and health. It is circumferentially covered by dense, hard cortical bone which imparts significant mechanical strength. It is an optimal structure in which to place an anchor, e.g., pedicle screw. The size and length of the implant is dependent upon the size and length of the cancellous core. See FIGS. 3A and 3B for an anatomic illustration of pedicle anchor placement in a vertebra. However, the posterior origin, i.e., “os”, of vertebral pedicle is obscured by native bone and in many cases, abnormal tissue related to spondylosis, all of which disrupt normal posterior morphology.

2. Photoacoustic Imaging

Photoacoustic imaging (PAi) delivers laser light to biological tissue, where energy from the laser light is absorbed by the biological tissue and converted to heat, which produces photoacoustic signal, i.e., an ultrasonic emission. The ultrasonic emission can be captured by an acoustic sensor such as an ultrasound transducer and analyzed to determine a composition of the biological tissue (e.g., bone, nerve, blood vessel, and/or the like).

It is well known in the medical literature that the laser light will be transmitted, reflected, scattered, auto-fluoresce, and absorbed differently among different human tissues, e.g., skin, adipose, muscle, tendons, ligaments, bones, blood, inorganic products of metabolism such as kidney stones, contingent upon selectable and adjustable laser light parameters like wavelength, energy, fluence, pulse-width, frequency, and target chromophore within the tissue or inorganic compound. In the case of PAi, the light absorbed by the target chromophore heats up the tissue, causing it to expand and, in the process, generate a pressure (acoustical) wave which radiates away from the chromophore location. That location can then be precisely detected externally by acoustic sensor(s) and displayed on an ultrasound monitor.

The utility of PAi in bone is supported by Thella et al., J. Orthop., 2016, 13(4):394-400; He et al., Comparison study on the feasibility of photoacoustic power spectrum analysis in osteoporosis detection, In Photons Plus Ultrasound: Imaging and Sensing, International Society for Optics and Photonics, 2017, vol. 10064, p. 100645H; Larshkari and Mandelis, Journal of Biomedical Optics, 2014, 19(3):036015; and Shubert et al., Phys. Med. Biol., 2018, 63:144001.

PAi can be used to penetrate mammalian calcified and/or non-calcified (soft) tissues. The tissues can be normal tissues or abnormal tissues such as tissues with disease or injury.

In some embodiments, the calcified tissue is mammalian cortical and/or cancellous bone. In some embodiments, the non-calcified tissue is a soft tissue such as skin, adipose, muscle, blood, nerve, or connective tissue.

The laser parameters, such as laser wavelength, power, pulse-width, fluence, frequency, position, and orientation (trajectory), can be selected to detect quantifiable depths of an acoustical return signal generated within the calcified and/or non-calcified tissues, especially within cancellous bone.

In some embodiments, the laser wavelength is selected as the absorption wavelength of a constituent of the tissue under measurement. For bone, the laser wavelength can be selected as the absorption wavelength of an organic constituent, e.g., blood, or an inorganic constituent of bone, such as hydroxyapatite and calcium phosphate. For soft tissues, laser parameters such as wavelength, fluence, frequency can be selected to interact preferentially with endogenous elements of the tissues, e.g., oxyhemoglobin, deoxyhemoglobin, lipids, water, etc., to alter the quality of the photoacoustic signal in predictable and quantifiable ways, such as depth of penetration, spatial resolution, etc.

In some forms, the methods further include utilizing one or more systemic contrast agents as a primary chromophore to accommodate different laser wavelengths with the effect of modifying or amplifying the strength of the acoustic response, e.g., increasing the signal-to-noise ratio, etc. Exemplary contrast agents include methylene blue, indocyanine green (ICG), noble metal nanoparticles, etc. See Luke et al., Annals of Biomedical Engineering, 2012, 40:422.

3. Use of PAi in Spinal Surgery

As described above, cancellous bone is blood rich and porous, in stark contrast to cortical bone which contains little blood and is dense. All human vertebrae are composed largely of cancellous bone which is covered by a relatively thin layer of cortical bone. By selecting laser parameters capable of passing through cortical bone and being preferentially absorbed by constituents and/or structure unique to the histology of cancellous bone will stimulate an acoustical wave unique to it relative to cortical bone. Finding the vertebral pedicle starting point on the basis of the foregoing lies in a unique methodology based upon morphologic features of all vertebral subtypes, i.e., lumbar, thoracic and cervical.

Structures common to all human vertebrae are the following posterior structures: the lamina (bilaterally), superior and inferior facets (bilaterally), and transverse processes (bilaterally) (FIGS. 1, 2A, and 2B). These structures converge and are continuous with a morphologic “feature” commonly referenced as pars interarticularis (FIGS. 2A and 2B). Thereunder, the only bony structures are the vertebral pedicles. As the pedicles ascend anteriorly, they unite with a large ovoid structure, the vertebral body (FIG. 1).

The outer layer of cortical bone covering the pedicles is continuous with the vertebral body. The cancellous core of the pedicles is continuous anteriorly with cancellous bone comprising the vertebral body, and with the aforementioned posterior spinal column structures (FIG. 4). The pedicle is bound medially by the spinal canal through which the spinal cord, nerve roots, and vessels pass, and laterally by soft tissues, i.e., muscle, ligaments, tendons, vessels, connective tissue, etc., and superiorly and inferiorly by nerve roots exiting through neural foramen formed by the superior and inferior facets of opposing vertebrae (FIG. 1).

The longest uninterrupted column of cancellous bone comprising the posterior, middle, and anterior spinal columns in all human vertebrae is one originating at or in the region of the pars interarticularis of the posterior column continuing up through the pedicle and continuous with the cancellous bone comprising the vertebral body in the anterior column (FIG. 4).

As such, the pars interarticularis is a reliable anatomic landmark to direct a beam of laser light at or position a fiber that diffuses laser light over to locate the posterior origin, i.e., os, of the pedicle. In some embodiments, the parameters of the laser light are selected to penetrate a single layer of cortical bone covering the pars interarticularis into cancellous bone thereunder, reaching a quantifiable depth within the cancellous bone. The parameters of the laser light include wavelength, power, frequency, pulse-width, fluence, position, orientation (trajectory), etc.

In some embodiments, the quantifiable depth covers at least the full length of the pedicle. In some embodiments, the quantifiable depth extends beyond the length of the pedicle and further into the cancellous bone of the anterior vertebral body of the vertebra. In some embodiments, the quantifiable depth may engage the anterolateral cortical bone of the vertebral body.

The PAi data can be used to locate an intra-cancellous pedicle implant path. In general, the intra-cancellous pedicle implant path starts from an intact cortical bone surface overlying the pedicle os, and extends through the cancellous bone of the pedicle into the cancellous bone of the anterior vertebral body. In some embodiment, the intra-cancellous pedicle implant path resides within the three consecutive vertebral columns (i.e., posterior column, middle column, and anterior column), as illustrated in FIGS. 3A and 4.

In some embodiments, the intra-cancellous pedicle implant path is pre-determined by a computed tomography scan, such as a computed axial tomography scan (FIG. 5). Alternatively, the quantitative PAi data can be incorporated into an image guidance platform wherein the necessary penetration depths by PAi can be superimposed over the preoperatively placed line segments denoting the length of individual vertebral pedicle lengths.

In some embodiments, the trajectory of the laser light overlaps with the intra-cancellous pedicle implant path. In some embodiments, the trajectory of the laser light does not overlap with the intra-cancellous pedicle implant path.

In some embodiments, the methods further include selecting and/or adjusting parameters of the laser light to penetrate surrounding soft tissues of the vertebra (especially around the pars interarticularis region), including skin, adipose, connective tissues, muscle, tendons, ligaments, etc., for tissue delineation. This can provide valuable information to identify the precise location of the pedicle os.

Based on published lengths of vertebral pedicles and reliance upon the pars interarticularis as a landmark to localize the pedicle os, finding an intra-cancellous pedicle implant path, which originates from the pedicle os and extends through the length of the pedicle, is within the limits of PAi. The published lengths of vertebral pedicles can be found in the following references: Grivas et al., Scoliosis and Spinal Disorders, 2019, 14:2; Cruz et al., Int. J. Morphol., 2011, 29(2):325-330; Kayalioglu et al., Neurol Med Chir, 2007, 47(3):102-107; Karaikovic et al., J. Spine Disord, 2000, 13(1):63-72; Frederick, Journal of Anatomical Sciences, 2015, 6:2; Zindrick et al., SPINE, 1987, 12:160-5.

Coincident with the foregoing method of finding the implant starting point, the same laser light can also be manipulated to measure the radial boundaries of the pedicle cancellous core, thereby providing an appropriate implant diameter not exceeding the boundaries of the cancellous core. The boundaries of the pedicle cancellous core can be determined by differentiating the cancellous bone in the pedicle cancellous core from the surrounding cortical bone. In some embodiments, the photoacoustic signals concerning the cortical bone appear as compact, high-amplitude signals, whereas the photoacoustic signals concerning the cancellous bone appear as diffused, low-amplitude signals. The measurement can also be performed in real time to guide drilling or cannulating in the pedicle (i.e., maintenance an optimal drill or cannulation trajectory) and/or placement of spinal implant.

The PAi data can be presented to the operator in multiple ways. In some embodiments, the photoacoustic data captured by the acoustic sensor can be converted into visual and/or audible cue(s) to indicate it is safe to commence drilling or cannulating. For example, a “go” signal could be denoted by a green “bull's eye” in a red field on a monitor when the correct location and angle of the drill is established on the cortical bone surface. In addition to presenting the data to the operator in a simple and meaningful form as per the aforementioned example, the data can be collected and processed by an computer algorithm which can correlate morphologic, anatomic, and histologic features of cancellous bone common to pedicles at different levels, disease states, etc., and thus enable real-time robotic “hands-free” spinal implant placement.

4. Exemplary Methods

Disclosed herein are exemplary methods for using photoacoustic imaging to locate vertebral pedicle starting point and/or optimize implant trajectory for spinal fusion surgery in a subject. The methods include: (a) directing a beam of laser light at or positioning a fiber that diffuses laser light over a pars interarticularis of a vertebra of the subject; and (b) acquiring photoacoustic signal for imaging the vertebra. In some embodiments, the photoacoustic signal is collected by an ultrasound transducer. FIG. 6 illustrates the region wherein the bean of the laser light is positioned or diffused. In some embodiments, the methods further include, prior to step (a), surgically exposing the posterior of the vertebra.

In some embodiments, the methods further include selecting and/or adjusting parameters of the laser light such that the laser light penetrates a single layer of cortical bone covering the pars interarticularis into cancellous bone thereunder, reaching a quantifiable depth within the cancellous bone. The parameters of the laser light can include wavelength, power, pulse-width, fluence, position, orientation (trajectory), etc. FIG. 7 illustrates exemplary laser trajectories.

In some embodiments, the quantifiable depth covers at least the full length of the pedicle. In some embodiments, the quantifiable depth extends beyond the length of the pedicle and further into the cancellous bone of the anterior vertebral body. In some embodiments, the quantifiable depth may engage the anterolateral cortical bone of the vertebral body.

In some embodiments, the methods further include locating an intra-cancellous pedicle implant path based on the photoacoustic signal from step (b). The intra-cancellous pedicle implant path starts from an intact cortical bone surface overlying the pedicle os, and extends through the cancellous bone of the pedicle into the cancellous bone of the anterior vertebral body.

In some embodiments, the intra-cancellous pedicle implant path is pre-determined by a computed tomography scan, such as a computed axial tomography scan, prior to step (a). FIGS. 8A and 8B show an exemplary vertebra and its computed axial tomography image.

In some embodiments, the methods further include selecting and/or adjusting parameters of the laser light and/or ultrasound transducer (e.g., >20 kHz) to penetrate surrounding soft tissues of the vertebra (especially around the pars interarticularis region), including skin, adipose, connective tissues, vessels, muscle, tendons, and ligaments for tissue delineation.

In some embodiments, the laser wavelength is selected as the absorption wavelength of an inorganic constituent of bone, such as hydroxyapatite and calcium phosphate.

In some embodiments, the laser parameters such as wavelength, fluence, and frequency can be selected to interact preferentially with endogenous elements of one or more of the surrounding soft tissues, e.g., oxyhemoglobin, deoxyhemoglobin, lipids, water, etc., or exogenous elements, the so-called “contrast agents”, e.g., methylene blue, indocyanine green (ICG), nanoparticles, etc., to alter the quality of the photoacoustic signal in predictable and quantifiable ways, such as depth of penetration, spatial resolution, etc.

In some embodiments, the methods may include imaging the vertebra with more than one set of parameters of the laser light and/or more than one set of parameters for the ultrasound transducer. This can generate different images with different degrees of tissue delineation. For example, some images may have better visualization of the bone structures of the vertebra, and some other images may have better visualization of the surrounding soft tissues. Taken together, this approach can improve the precision in locating the pedicle os and the intra-cancellous pedicle implant path.

In some embodiments, the methods further include, after step (b), transmitting the photoacoustic signal to a visual form on a monitor, and/or to an audible form, which optionally changes pitch and/or volume based upon proximity to the vertebral pedicle starting point for guiding surgical operation.

In certain embodiments, the methods further include drilling along the intra-cancellous pedicle implant path under the guidance of the photoacoustic imaging data obtained from step (b). The methods may also include installing the implants, such as pedicle anchors, after drilling.

Unless defined otherwise, all technical and scientific terms used herein have the same meanings as commonly understood by one of skill in the art to which the disclosed invention belongs. Those skilled in the art will recognize, or be able to ascertain using no more than routine experimentation, many equivalents to the specific embodiments of the invention described herein. Such equivalents are intended to be encompassed by the claims.

### III. Devices and Systems

The PAi devices and/or systems for performing the disclosed methods are known in the art. Examples can be found in U.S. Patent Application Publication No. 2015/0223903 and Shubert et al., Phys. Med. Biol., 2018, 63:144001. The PAi devices and/or systems can be configured for use in a spinal surgery, especially spinal fusion.

The PAi devices and/or systems generally include a laser source, optionally coupled with an optical fiber, and an acoustic sensor, such as an ultrasonic transducer, preferably positioned at or near a site of surgical procedure. In certain embodiments, the ultrasound transducer is configured to acquire B-mode images.

The devices and/or systems can also include a non-transitory computer readable medium configured to process the photoacoustic data. In certain embodiments, the non-transitory computer readable medium is programmed to execute coherence-based beam forming to correct for insufficient laser fluence.

The devices and/or systems can also include a medical device such as a surgical tool.

The devices and/or systems can also include or be coupled with a robotic system. The robotic system can be used to control the laser source, the optical fiber, the acoustic sensor, the medical device, or a combination thereof. The robotic system can be controlled by the foregoing non-transitory computer readable medium and/or by a second non-transitory computer readable medium.

The devices and/or systems can also include image quality/performance metrics used to ascertain information for guiding surgical procedures.

In some forms, the devices and/or systems contain (1) a tracking module which includes a laser source, optically coupled with an optical fiber, wherein the tracking module generates tracking data, (2) a photoacoustic module which includes an acoustic sensor, wherein the photoacoustic module is configured to acquire the photoacoustic data, and (3) a computing module which includes a non-transitory computer readable medium, wherein the non-transitory computer readable medium is programmed to process the tracking data and the photoacoustic data using coherence-based beam forming (e.g., SLSC). The acoustic sensor can be an ultrasound transducer or a photoacoustic-module optical fiber. In some embodiments, the photoacoustic-module optical fiber can also be the optical fiber of the tracking module. In some embodiments, the tracking module can be coupled to the photoacoustic module.

1. A method for using photoacoustic imaging to locate posterior os of a pedicle on a vertebra and/or optimize implant trajectory for spinal fusion surgery in a subject, comprising:
(a) directing a beam of laser light at or positioning a fiber that diffuses laser light over a pars interarticularis of the vertebra of the subject; and
(b) acquiring photoacoustic signal for imaging the vertebra.

2. The method of claim 1, wherein the photoacoustic signal is acquired by an ultrasound transducer.

3. The method of claim 1, further comprising, prior to step (a), surgically exposing the posterior of the vertebra.

4. The method of claim 1, further comprising selecting and/or adjusting parameters of the laser light such that the laser light penetrates a single layer of cortical bone covering the pars interarticularis into cancellous bone thereunder, reaching a quantifiable depth within the cancellous bone.

5. The method of claim 4, wherein the parameters of the laser light comprise wavelength, power, pulse-width, fluence, position, orientation (trajectory), and combinations thereof.

6. The method of claim 4, wherein the quantifiable depth comprises at least the full length of the pedicle.

7. The method of claim 6, wherein the quantifiable depth extends beyond the length of the pedicle and further into the cancellous bone of the anterior vertebral body of the vertebra.

8. The method of claim 1, further comprising locating an intra-cancellous pedicle implant path based on the photoacoustic signal from step (b), wherein the intra-cancellous pedicle implant path starts from an intact cortical bone surface overlying the pedicle os, and extends through the cancellous bone of the pedicle into the cancellous bone of the anterior vertebral body of the vertebra.

9. The method of claim 8, wherein the intra-cancellous pedicle implant path is pre-measured from a computed axial tomography scan, prior to step (a).

10. The method of claim 1, further comprising, after step (b), transmitting the photoacoustic signal to a visual form on a monitor and/or an audible form which changes pitch and/or volume based upon proximity to the vertebral pedicle starting point for guiding surgical operation.

11. The method of claim 1, wherein the laser wavelength is selected as the absorption wavelength of an inorganic constituent of bone.

12. The method of claim 11, wherein the inorganic constituent of bone is hydroxyapatite.

13. The method of claim 1, further comprising utilizing one or more systemic contrast agents as a primary chromophore to accommodate different laser wavelengths with the effect of altering the photoacoustic signal.

14. The method of claim 13, wherein the systemic contrast agent is methylene blue, indocyanine green, or noble metal nanoparticles.

15. The method of claim 1, further comprising selecting and/or adjusting parameters of laser light such that the laser light penetrates surrounding soft tissues of the vertebra to discern morphologic features of the vertebra.

16. The method of claim 15, wherein the soft tissues comprise skin, adipose, connective tissue, muscle, ligaments, tendons, or a combination thereof.

17. The method of claim 15, further comprising selecting and/or adjusting parameters of the ultrasound transducer to image the soft tissues.
